(19) United States
(12) Patent Application Publication
(10) Pub. No.: US 2023/0061486 A1
(43) Pub. Date: Mar. 2, 2023

(54) DETERMINATION OF QUALITY AND ORIGIN OF FISH BY EPIGENETICS

(71) Applicant: TampoGene AS, Oslo (NO)

(72) Inventor: Øystein LIE, Oslo (NO)

(21) Appl. No.: 17/796,024

(22) PCT Filed: Jan. 29, 2021
(86) PCT No.: PCT/NO2021/050030

§ 371 Date: Jul. 28, 2022

(30) Foreign Application Priority Data
Jan. 31, 2020 (NO) 20200119

(51) Int. Cl. C12Q 1/6888; A01K 61/10

(52) U.S. Cl. CPC C12Q 1/6888; A01K 61/10; C12Q 2600/124; C12Q 2600/154

(57) ABSTRACT

The present invention relates to aquaculture, fish farming, to fish production and particularly smolt production, to traceability of fish and resulting seafood products, and to a method for providing robust and high-quality farmed fish. More particularly, the invention provides a method to identify farmed fish characteristics, comprising a step of preparing epigenetic signatures of the fish. The epigenetic signatures are employed for tracking origin and identity, as quality verifiers, as predictor for sea phase performance, as well as for feedback markers to optimize production regimes.

### FIELD OF THE INVENTION

The present invention relates to aquaculture, and particularly to aquaculture of fish, smolt production of salmonid fish included. Further, the invention relates to methods for traceability of fish, seafood products, and to a method for providing robust and high-quality farmed fish.

### BACKGROUND OF THE INVENTION

Although fish farming, and particularly the aquaculture of salmonid species, is considered as a successful young industry in many temperate countries, with Norway as a leading player, there are still many challenges. Several of these challenges are related to fish health and welfare and to environmental issues, and the challenges have considerable impact on the economics and the sustainability of the sector. Major components of the problem are infectious diseases, parasite infestations like sea lice, developmental malformations, highly variable growth rates in the sea phase, and adverse environmental impacts including fish escapes, wastes, transfer of parasites and infections to wild stocks.

Further, salmon industry is still immature in the sense that most of the marketing volume is in the form of bulk products and less as value-added and branded seafood. This means that this quite volatile/price sensitive sector with very delicate production regimes in the pre-harvest part is not taking full advantage of the margin opportunities which could have been enjoyed with branded higher priced products. Moreover, the existing brands are so far not protected sufficiently by biological markers verifying their origin.

Presently, the above-mentioned problems have been sought to be solved through multiple sets of preventive and curative regimes. However, systemic problems need system solutions where the one measure does not exclude the other and may consist of: multiple sets of preventive and curative regimes. These may consist of rules and regulations from authorities linked with permits/licenses (quarantines, max admitted biomass, locations), fish welfare and best practice managements, breeding, vaccines, feeds and feed ingredients, treatments, optimized gears and infrastructure, optimized managements. Despite of all these efforts, the fish farming sector is still suffering from the described challenges, some of which even have escalated over the recent years.

One part of the above described complex problem is related to inferior seed quality, i.e. smolt in salmon farming. The smolt is often not as robust as desired, mostly due to suboptimal smolt production regimes.

Moreover, the existing test systems aimed at reflecting robustness, maturation, life phase readiness etc. in fish farming, such as in the smolt production, are inferior. For instance, the current methods to evaluate the entire smolt production regimes, critical phases included like the smoltification process (a metamorphic process) and smolt window timing, are not robust enough simply due to the fact that they are restricted to the testing of single or too few biological markers compared to the complexity of the mentioned biological processes. The nature of the fish and smolt quality is resulting from a vast number of systems' biology interplaying factors and pathways under varying genetic control and environmental influence, the latter also embracing production regimes with accompanying protocols. As for the smoltification part; not only are a lot of bioelements and biorhythms involved but many of them also need to be synchronized. Hence, there is a need for an analytical method that can reflect the fish maturation status and robustness in its various life phases in a better way, and also which in turn is able to provide feedback to optimizing the fish production and accompanying management regimes in such.

### BRIEF SUMMARY OF THE INVENTION

The invention provides methods for farmed fish handling or production comprising the provision and analysis of epigenetic signatures of fish sample materials. The invention provides methods to identify farmed fish characteristics or its origin, by the provision and analysis of epigenetic signatures of fish sample material.

Hence, by the provision and analysis of epigenetic signatures of fish sample materials one can identify different fish production regimes with their different fish characteristics or their origin, location included, since location will be interlinked both with production regime and with unique environmental characteristics with impact on the epigenome.

The method comprises the steps of correlation analysis between either of epigenetic signatures; epigenetic signatures and fish performance, robustness or health; or epigenetic signatures and production protocols.

The provision of the epigenetic signatures and the correlation analysis may be used in the verification of fish robustness and health and resulting product quality; in feedback to the fish farming production; as an authenticator and verifier of origin e.g. to assist in building and protecting brands; or for detecting origin of cultured fish, such as detecting origin of escapees.

Existing tracking systems based on brood stock and pedigree information have a restriction on assigning to locations and production regimes opposed to epigenetic profiles which are strong reflectors of such.

In one aspect the invention provides a method to identify fish characteristics of farmed fish, comprising a step of preparing at least one epigenetic signature from a fish sample material, wherein the method comprises the steps of:
i) sampling to obtain fish sample material;
ii) DNA sequencing, comprising carrying out genome sequencing of the fish sample material;
iii) analysing the revealed genome data set of step ii) and establishing epigenetic signatures for the samples; and optionally
iv) comparing and correlating the epigenetic signatures obtained with existing epigenetic signatures;

wherein the prepared epigenetic signatures of the fish sample material are for use as authenticators for fish.

Such use may include in traceability of fish, for use as a verification of a given fish production protocol/regime, such as for use in determination of the origin of escaped farmed fish.

### BRIEF DESCRIPTION OF THE DRAWINGS:

FIG. 1 is a scatter plot of average methylation values per gene and per organ, with data from 53 715 gene boundaries combined for liver and kidney, prepared from samples of smolt of cultured Atlantic salmon.

FIG. 2 is a frequency distribution graph based on the same number of genes as for FIG. 1, and from the same samples as used for FIG. 1, are sorted by methylation values, wherein methylation values for kidney are dark grey, for liver are white and where the global picture, the data of the two tissues merged, is light grey.

FIG. 3 provides a scatter plot of selected genes, from the same samples as used for FIG. 1, with a large difference in methylation value together with the global picture, and including 6583 genes, using an arbitrary cut off, <0.2 AND >0.8 in both the liver and kidney tissues, also including genes which are unmethylated in both tissues.

FIG. 4 is a scatterplot of a collection of 46 genes, from the same samples as used for FIG. 1, belonging to the Hox gene family, large black dots, based on data from both the liver and kidney tissues and from the total pool (53 715) of genes.

FIG. 5 is an illustrative sketch of the concept of the invention, comprising sampling, generating epigenetic signatures and recording of performance data.

FIG. 6 provides the genome-wide methylation pattern of salmon smolt using a window of 100,000 base pairs for two production regimes (RAS and Flow thorough), and differences between them.

FIG. 7 provides a graph of the development index (the inverse ratio of methylation frequency) from blindly analysing 5 groups of smolt from the same operator.

FIG. 8 provides a graph of the development index from analysing groups of smolt from two different operators.

FIG. 9 provides the CpG DNA methylation patterns at 2000 bp upstream regions of genes located on the chromosome “NC_027300.1” across five life stages of salmon.

FIG. 10 provides a violin plot demonstrating the distribution of methylation values for each gene/feature of salmon.

### DETAILED DESCRIPTION OF THE INVENTION

The method of the invention comprises the provision and analysis of epigenetic signatures of fish sample materials. Epigenetics refers to the study of heritable changes that do not involve changes to the DNA sequence, but which have resulted from chemical modifications to the DNA, chromatin remodeling, histone modification or noncoding RNA which affect gene expression. Epigenetic marks, or signatures, which act on expression both in time (“biological clock”) and space (tissue development), can be inherited to progeny of cells (epigenetic memory) or to progeny of organisms (transgenerational inheritance). In the former case it is a major driver of cell differentiation and the development of an individual's entire life span. In the latter case the marks are imprinted in the germ line genome (sperm or egg cells) of the parents and may bestow parent specific “messages” to govern the expression of selected genes of the offspring provided the marks overcome the gametogenic and embryonic reprogramming which normally takes place at a high degree. Part of the marks do overcome this reprogramming, which is the reason why epigenomes can display transgenerational inheritance. A considerable number of genes of vertebrates are differentially expressed in the offspring related to the parent of origin: a copy (allele) of a specific gene inherited from one parent may be expressed whereas the other allele of the same gene inherited from the other parent may be non-expressed. This parent of origin specific expression is called genomic imprinting. Appropriate imprinting of specific genes is important for normal development.

Epigenetic marks are intimately linked with the biorhythms of an individual and can change, i.e. become reprogrammed, in response to environmental stimuli over the course of an organism's life. The applicant has now found that details of the management regimes or environmental effectors during the pre-harvest fish farming phase affect the epigenetics and render epigenetic signatures in the organism, and this fact may be used in farmed fish handling or production, smolt production included.

One type of epigenetic mechanism is DNA methylation, where a methyl group (CH₃—) is added to bases of genomic DNA by specific enzymes, DNA-methyltransferases. Usually it is the base Cytosine (C) which is methylated at the same 5-position of the pyrimidine ring (5mC) or hydroxymethylated (5hmC), and most often the cytosine residue is followed by a guanine residue, forming a CpG site. Sequences enriched in CpG sites, called CpG islands, often surround promoters and are typical sites for transcription initiation. Methylation can change gene expression. When located in a gene promoter, DNA methylation typically acts to repress gene transcription. When located in the gene body, methylation may enhance gene expression, especially if the CpG islands in the promoter region are not methylated or hypomethylated. DNA methylation is essential for normal development and is associated with a number of key processes including genomic imprinting, X-chromosome inactivation, repression of transposable elements, aging, carcinogenesis, and cell differentiation.

Two of DNA's four bases, cytosine and adenine, can be methylated. At least for humans and other mammals, it is known that DNA methylation levels can be used to estimate the chronological age of tissues, cell types and individuals based on their biological age. The latter is obtained by correlating the shifting methylation pattern with time and cell divisions, and hence forming an accurate epigenetic clock.

Definitions:

By the term “methylome”, we mean the set of methylated modifications of the DNA of an organism's genome or cells. Methylation is a major chemical change within the term epigenome (see below) to change the function of a genome.

By the term “epigenome”, we mean chemical changes to the DNA and histone proteins of an organism that can be passed down to progeny of cells and organisms. Such changes can lead to functional changes of the genome like influencing gene expression, while the term “transcriptome” means the set of RNA molecules in a cell at a given point of time or the full range of messenger RNA molecules expressed by an organism. The term includes both amount and identity of specific RNA molecules.

Further, with the term “methylome signatures” or “epigenetic signatures” we mean the pattern with which the methylation is distributed in the genome of a cell or an organism when the epigenetics records are restricted to methylation records, and this may also be called a “DNA methylation profile”. The signatures or profiles may be defined in several distribution dimensions: by organ/tissue, by life phase, by genome segment (chromosome), by gene, or by CpG site or by CpG island. The terms “epigenetic signatures”, “methylome signatures” and “DNA methylation profile” have more or less the same meaning since methylation is a major factor of epigenomes and hence the terms are used interchangeably herein.

By the term “gene expression profiles” we mean a collection of a series of transcripts of specific genes as of identity and amount and by the term “transcriptome profiles” we mean expression patterns of the transcriptome studied at cell or specific gene level.

The term “smolt window” is defined as the critical time period where the captive smolt has to be transferred to sea and the wild smolt has to swim downstream and successfully adapt to saltwater to avoid desmoltification; reversal of the smoltification process. Desmoltification will normally cause massive death if transferred under this status. The smolt window is substantially influenced by environmental factors both in culture and in the wild, such as temperature, photoperiod, salinity.

By “smolt status”, we mean status of maturation, readiness for transfer to sea, if evaluated in the smolt window, and general robustness.

By “sea phase”, we mean the period from transfer to sea to harvest and by “sea phase grow-out performance” we mean fish characteristics such as, but not limited to, survival rate, growth rate, specific health or disease records.

By “post-harvest characteristics”, we mean carcass qualities such as meat texture and colour.

By the term “robust smolt”, we mean smolt which in the sea phase displays relative superior records in terms of growth and survival rate.

By the term fish characteristics, we mean welfare traits like fish robustness, health, growth rate, behaviour, appetite, as well as various product qualities like texture and colour. There is a series of subgroups of these characteristics along with the whole lifespan and value chain which may be grouped into welfare needs and welfare indicators both on group, as well as on individual level and guided by The Food Safety Authority and regulated by the Animal Welfare act (in Norway) and similar authorities and regulations elsewhere.

The present invention provides a solution to the problems related to inferior production regimes of farmed fish and particularly to smolt, and also to the problem of traceability of farmed fish, by the use of epigenetics. The provision and analysis of epigenetic signatures of samples of farmed fish according to the method of the invention, can be used in:

the verification of fish robustness, health and resulting product quality;

in feedback to the aquaculture production, such as the fish farming production, e.g. for amending or optimization of such;

as an authenticator or verifier of origin, e.g. to assist in building and protecting brands, or for detecting origin of cultured fish, such as detecting origin of escapees.

The applicant has found that a method, or a process, comprising the provision of epigenetic signatures of fish, which may be accompanied with gene expression profiles, can be used as objective biomarker reflectors of fish characteristics, e.g. to achieve any of the above-mentioned applications.

The disclosed and claimed methods take advantage of knowledge and facts about the vertebrate epigenome. The concept of the invention employs epigenetic profiling to identify fish characteristics, such as to further verify and optimize farmed fish production quality. The disclosed method is useful for farmed fish wherein this is from the group of bony fish (teleost). Particularly, the method may be used for teleost fish species that are farmed commercially, and particularly for salmonid species. The fish is e.g. selected from the group comprising Atlantic salmon and brown trout (Salmo salar and Salmo trutta respectively), Steelhead/Rainbow trout, Chinook salmon, Coho salmon and other Pacific salmonid fishes (Oncorhynchus s.p.p.). In one embodiment, the fish is from a non-salmonid fish group, such as tilapia, catfish, sea bass or sea bream.

For the numerous teleost species (almost 30 000 known), a series of which also is subject to long standing aquaculture, there are diverse modes of development both in the wild and in captivity. This is due to plenty of time for evolvement and radiating into 45 orders and over 435 families adapted to a lot of different habitats and ecosystems in saltwaters, freshwaters as well as in both. Of the anadromous fish like salmonids, some migrate between fresh and saltwater and some stay landlocked. Sex and sex development in fish displays high degree of plasticity which means that although governed by genetic mechanisms, sex can develop alternatively male or female direction also depending on environmental effectors like temperature, feeding, density, social factors, pH, oxygen concentration etc. Some species are hermaphroditic, some are unisexual (all female based on natural gynogenesis), some change sex during life span. The general explanation to this heterogeneity and plasticity is the absence of single genetic sex determining genes or single genetic cascade but rather a flexible system of polygenes interacting with a lot of environmental factors and linked up with a similarly heterogenic sex chromosomal structure. This implies that many aquaculture operators have taken the advantage of employing a variety of reproductive and clonal or chromosome manipulation techniques (like gyno- and androgenesis) to develop all one sex fish, sterile fish (e.g. triploid), and isogenetic hybrids.

The salmonids go through certain lifecycles, both in captivity and in the wild: When a fertilized egg is ready to hatch, the embryo or juvenile will break free from the egg's soft shell retaining the yolk as a nutrient-rich sac. At this stage, they are called Alevins, or yolk sac larvae. The stage at which the yolk sac has disappeared, and the juveniles have become capable of feeding themselves they are called fry. From start feeding point in time and during an on growing period, depending particularly on management parameters like temperature, they eventually develop into parr with camouflaging vertical stripes. The transformation from parr to smolt is in developmental terms quite fundamental, like a metamorphosis. It is then adapted to a life of drastic contrasts to the existing through vast changes in morphology, salt tolerance, metabolism, behaviour. During this transformation, parr marks fade, fin margins darken, and the body becomes more streamlined with a bright, silvery appearance. In both captivity and in the wild a major behaviour change is the swim pattern, shifting from moving against the stream to swimming downstream. Early life in the wild (river) spans from 2 to 5 years after which the fish migrates long distances in the ocean, feed and sexually mature within 1-4 years and re-enter home river for spawning. The spawners may range in size from 2 to above 15 kg and sexual maturity is the signal for turning home. Genetics, feed availability and environmental factors are influencing both time to sexual maturity as well as ocean growth rate. The overall returning percentage (survival) is low (approx. 2%) emphasizing strong selection for fitness. The homing success (percentage of the survivals returning to home river) is however much higher (likely between 70 and 90%) in order to maintain environmental adaptation and specialisation, but some migration to other rivers also leave room for hybridization to avoid inbreeding and hence enhance or maintain genetic variation and robustness.

In fish farming and production of fish in captivity, there is a variety of production regimes, and particularly smolt production regimes, wherein the main distinguishing parameters are: temperature and photoperiod structures, the type of system used such as throughflow systems or recirculation systems (RAS), feeding regimes, salt adaptation etc. These parameters also have great impact on the time from hatching or start feeding to smoltification. From the fact that observed reduced robustness and malformations seem to have become an increasing problem in the grow out phase, there is reason to believe that high throughput regimes running with relative high temperatures, such as e.g. over 8° C. at the fertilized egg phase and yolk sac phase, and over 12° C. at the start of feeding of fry, will bestow increased incidences of backbone deformities. One cannot exclude that nature itself has a better solution, leaving the early fragile life in the river at low winter temperature and also leaving the fry to stay and mature for much longer period (e.g. 2-5 years) in fresh water than the commercial regimes which range from less than 1 year to 2 years.

The commercial sea phase grow-out period for Atlantic salmon is currently at an average of 12-14 months until culling at about 5-6 kg. Due to a series of measures over several decades the farming period has been reduced from about 2 years to the current time span. The major measures for this progress are: selective breeding for growth rate and delayed sexual maturation (the latter which otherwise would interfere with growth), feeding regimes, disease control regimes and a series of management.

It is not straight forward to determine the degree of maturation of fish, not least the complex multifactorial smoltification process in salmonid fish accompanied with the challenge of timing of the transfer from freshwater to sea. Also, as described above, sexual maturation, which is unwanted in cost-efficient farming, is a complex trait subject to high plasticity and interaction of genetic and environmental factors. Currently, there are several indicators that may be used in the assessment of maturation, such as smolt maturation, however due to the complex interplay between these, it is not sufficient to test single parameters, such as e.g. analysing the amount of certain proteins or gene transcripts. An epigenetic approach, however, as provided by the method of the invention, will cover necessary and informative biomarkers to reflect maturity and robustness status of importance for optimizing aquaculture of teleost fish, such as in production of farmed fish. Regardless of the fish species, variation in life-cycle, biorhythm, maturation, sex development, being natural or artificially imposed or linked with aquaculture management regimes, all developmental phases and effectors implemented will be reflected in the epigenetic signature, and this is used in the method of the invention.

The applicant provides a method wherein epigenetic signatures are obtained and analysed. The method comprises the following steps:
i) Sampling to obtain fish sample material;
ii) DNA Sequencing comprising carrying out genome sequencing, e.g. high through-put sequencing, of the fish sample material;
iii) Analysing the genome data set of step ii) and establishing epigenetic signatures for the samples; and optionally
iv) Comparing and correlating the epigenetic signatures obtained with existing epigenetic signatures, or alternatively or additionally correlating any of the prepared epigenetic signatures with performance data.

In more detail, the method hence comprises the following steps:
i) Sampling steps: The sampling steps include the collection of fish sample material, and this may be from any of individuals, organs, tissues or blood from any of the stages of the fish' life-cycle which may be used for genome sequencing. The steps comprise the collection of fish sample material, comprising any of early phase whole individuals (eggs or larvae), or selected organs, tissues, or blood from later or several phases of the individual fish. The samples may include fertilized eggs, larvae, fries, parr and individuals at smoltification stages (for salmonid fishes), and ultimately also later from the sea grow out phase until harvest or post-harvest, or organs, tissues, or blood from any of these. The samples may further include material for freezing as well as for RNA stabilisation, the former for methylome analysis and the latter for transcriptome analysis. The fish sample material is hence from any of individuals, organs, tissues or blood from any of the stages of a fish' life-cycle, comprising either of fertilized eggs, larvae, fry, parr and individuals at smoltification stages, from the sea grow out phase until harvest or post-harvest, or organs, tissues, or blood from any of these.
ii) DNA Sequencing steps: The steps comprise carrying out genome sequencing steps of the fish sample material, such as a full genome sequencing, to obtain a genome data set. These steps may include sequencing of the sampled individuals and/or relevant organs, tissues, or blood. These method steps further optionally, but preferably, comprise the step of comparing the obtained genome data set of the fish sample material with an existing genome DNA sequence for the fish. In one embodiment, this is a two-step (bioinformatics assisted) process where the first step comprises alignment of generated sequences with existing reference genomes to pull out annotated genes, followed by a second step where information is obtained about how DNA is methylated within, in the vicinity of, or more distant from certain genes. Reference is made to Example 1. In one embodiment, bioinformatic program software packages, e.g. such as Burrows-Weeler (BWA) and/or NANOPOLISH, are used for these steps of comparing the obtained genome data set with an existing genome DNA sequence, including obtaining information about gene boundaries and information about the methylation. This may further include a step of isolating RNA, either from specific target genes or genome wide.
iii) Analysing steps: The steps comprise the analysis of the epigenome, i.e. methylome, of the revealed genome data set and establish epigenetic signatures, such as either or both global genome-wide methylation analysis as well as specific epigenetic signatures. The epigenetic or methylome signatures may be defined in the context of, but not limited to either of: organ/tissue, cell types, genome segment (chromosome), gene and gene related structures, genome regulatory elements (e.g. promoter, enhancer), CpG sites, CpG islands. In one embodiment of the method, the epigenetic signature, and particularly the methylation distribution, of CpG islands is analysed. These may be displayed as e.g. scatter plots, employing contemporary sequencing technologies (e.g. Oxford Nanopore Technologies as described in Example 1), and adequate bioinformatic software both for methylation recognition as well as for displaying the methylation patterns. Additionally, one may also establish gene expression profiles or transcriptome profiles to assist in identification of genes involved together with their level of expression. The methylome signatures and added gene expression profiles may be related to the life phase of the individual, to specific organs, tissues or cells, or to specific candidate genes. Gene expression profiles may be carried out by microarray-based analysis or other platforms (qPCR or direct RNA sequencing) and may be displayed as on/off expression or as quantity of expression, e.g. as heat maps).
iv) Comparing and correlating steps: In one embodiment, the established epigenetic signature of step iii) for the fish sample of step i) is compared with existing epigenetic signatures, such as with epigenetic signatures of an epigenetic signature data bank, e.g. as generated by the applicant. Further, this step may comprise the step of correlating any of the prepared epigenetic (methylome) signatures, and optionally the gene expression profiles or transcriptome profiles, with performance data. Such performance data may comprise data for either of:

Life phase, fish (e.g. smolt) management regimes and protocols, traits and performance, sea phase grow out performance, or post-harvest characteristics (carcass qualities). The group of data which the epigenetic signatures is correlated with is herein called “Performance data bank”. The steps preferably include the use of statistical methods and analyses. This may be used for e.g. displaying correlation between different epigenetic/methylome signatures, and/or between epigenetic signatures and fish characteristics such as production protocols and sea phase performance data. When comparing epigenetic signatures with performance data, the method preferably includes a step of statistical correction for different genetic background (e.g. brood stocks), or such correlation should be carried out for fish with the same genetic background.

In one embodiment, the method comprises use of the following groups of data:
a) The epigenetic signatures of the fish, and preferably combining this with the genome outputs, e.g. transcriptome or expression profiles;
b) Performance data for the fish; e.g. health/welfare and qualities, including e.g. growth rate, survival rate, health records or carcass qualities;
c) Production regime data, e.g. from protocols or manuals from the fish producers;
d) Observation data from production, i.e. true data from production, e.g. temperature, O₂, CO₂, salinity, osmolality, or turbidity etc.

The method hence provides a possible merger and comparison of major and critical data relevant for the farmed fish, e.g. combination of any of: Epigenetic signatures and expression profiles; Production regimes and accompanying protocols; and records of various parameters under monitoring.

For the method of the invention the epigenome of the fish is obtained, and this is combined with the genome outputs, e.g. transcriptome and expression profiles, to complete the biomarker structure. Preferably all putative and informative biomarkers of the fish are given room for exposure. The fish biology is hence extensively exposed about its status of welfare opposed to trying to describe it through restricted methods and tests. The method hence includes a sequencing-based whole genome approach.

The method steps may be carried out by employing adequate bioinformatics tools. The obtained epigenetic signatures, as well as results from the expression analyses, may form part of an “epigenetic signature data bank”, as generated by use of the method of the invention.

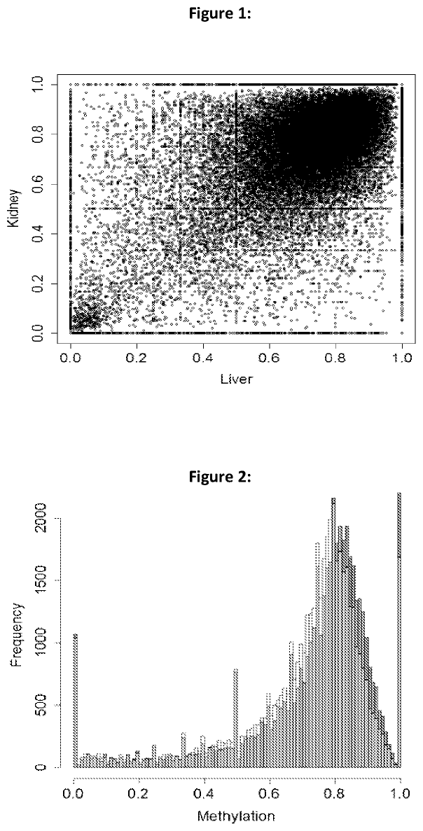

Examples of the sampling and sequencing steps (step i and ii), and analysis step (step iii) together with bioinformatic operations using an adequate set of bioinformatic software, are described in Example 1, and FIGS. 1 to 4.

For the sampling steps (i), material is sampled with a minimum sample size (i.e. minimum number of individuals) to ensure fish group representativeness which again is achieved by combining adequate statistics with information on degree of homogeneity of the group subjected to sampling. Preferably stratified sampling is performed, i.e. sampling from a population or a group. In the sampling step of the method, preferably at least one sample material, such as 1-100 fish sample material, such as 1-50 fish sample material, such 2-20 fish sample material, such as 2-10 fish sample material, such as 3-6 fish sample material, such as 4-5 fish sample material, are collected for each data collection. The sampling may further comprise a merger of samples from different individual fish. The sampling strategy should also be taking into account that fish may have different genetic background, e.g. are originating from different breeding regimes, and hence samples should be tagged for such background so as to account for this under step iv).

Further, for the sampling step, and e.g. for the accumulation of data for the epigenetic signature data bank, fish sample materials comprise material from either of:

Different life-phases of the fish' life-cycle, i.e. preferably from the fertilized eggs, larvae, fish fries, parr or individuals at the smoltification stages, sea grow-out phase until harvest or post-harvest;

Different organs, tissues or cells; e.g. sample material from liver, kidney, brain, guts or gills.

Bioinformatic mining (analysis) of the methylome data may generate the profile or the signatures by genome segments, e.g. chromosomal distribution patterns, by gene in terms of frequency and amount of methylation, and the same applies for selected candidate genes. The genome/gene database of the fish, such as of salmonid species, together with bioinformatic analysis, makes this possible, and the same goes for gene-specific signatures. Hence, one does not have to sample neither chromosomes nor genes but can distribute the methyl signatures on chromosomes and genes using bioinformatics, organ-based methyl data and the salmon genome bank. From this, in one embodiment, for the step of collecting fish sample material, its output signature reflects, either of, but not limited to;

different life-phases of the fish' life-cycle;

different organs, tissues or cells;

different genome segments, i.e. chromosomes; or selected genes.

Hence, in one embodiment, the method comprises a step of collecting fish sample material, wherein such samples are taken from, or its output signature reflects either of, but not limited to;

Genome: Chromosomal distribution, CpG islands, CpG sites, Gene Associated methylation, or Non-coding methylated areas;

Gene: Gene body, Gene promoter, Gene enhancer, Degree of methylation, or Frequency of methylated genes;

Organ, tissue or cell: Organ/tissue profile (individual organ/tissue, or several organs/tissues collected), Stem cell, Germ line cell, or Cells under differentiation (different phases, e.g. Biological clock at defined cell or defined tissue level), Differentiated cell (e.g. mature B-cells, T-cells);

Life phase (of the organism): Specific phase in life span profile, or the whole life-line profile (e.g. biological clock of the individual).

From the method of the invention, providing epigenetic signatures, and optionally gene expression profiles, these may be used for one or more of the following:
a) as authenticators for the fish; e.g. for use in traceability of fish. This may include the use as a verification of a given fish production protocol/regime, e.g. for a specific hatchery or smolt or fish farming production regime, such as for use in determination of the origin of escaped farmed fish.
b) to distinguish between different production regimes, i.e. different production regimes with accompanying protocols, and further distinguishing between sea phase performance and resulting product qualities from the different production regimes.
c) to provide feedback, e.g. to the hatchery and fish farming operators, to assist in optimizing the fish production protocols and regimes, such as the smolt production regimes, and/or the sea phase production regimes.
d) to predict sea phase grow-out performance based on smolt epigenetic signatures. E.g. making it possible to distinguish between smolt with different potentials for sea phase grow-out performance, or for predicting the resulting sea phase performance.
e) to verify either of quality or origin of the fish, such as to assist in brand building of the fish or smolt.
f) to determine or verify the degree of smolt maturation, such as to estimate the timepoint for end of the smolt window, i.e. the timepoint for transfer of the fish to sea water.

Hence, the knowledge obtained from the obtained epigenetic signatures, the “epigenetic signature data bank”, optionally correlated with performance date, the “Performance data bank”, may be used in a verification of the status or quality of a group of fish. Hence, in one embodiment of the method of the invention, a sample is obtained from a fish, at some stage in its life cycle, the epigenetic signature is obtained for this, and this epigenetic signature is compared with existing epigenetic signatures, such as of the epigenetic signature data bank, which for performance data exist, to link this e.g. to environmental conditions, such as to a given regime. Preferably, one should statistically correct for the effect of different genetic background of the fish or carry out comparison of signatures, and signatures and performance within fish of same genetic background.

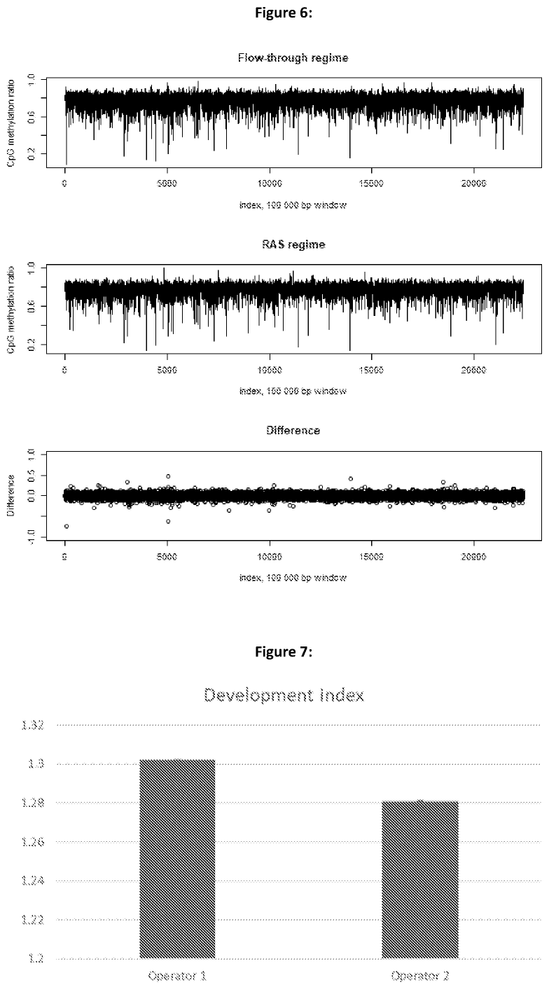

The applicant has compared the methylome signatures of smolt from different smolt production regimes. When comparing methylome signatures at the smolt window phase as well as gene specific methylation levels between different smolt production regimes (i.e. RAS and Flow through regime, respectively), and corrected for smolt size as well as genetic origin, unique patterns are revealed. In addition, strong contrasts between methylation levels for a series of genes have been found. This implies that the methylome contrasts between the regimes are induced by the measures linked with the regimes and not by genetic origin. FIG. 6 provides the genome-wide methylation pattern using a window of 100,000 base pairs for two production regimes (RAS and Flow through), and differences between them, reflected by the dots deviating from the clustered middle.

These findings again imply that regime and environment induced epigenetic variation is a novel tool that may be employed, potentially in addition to DNA fingerprint-based traceability, and should not be confused with the latter. Moreover, a series of genes of importance to e.g. iron ion homeostasis, antimicrobial activity, immune defense, gene regulation, development, maturation etc. were pulled out from this analysis, one of which is of particular importance is number three of the top list of Table A: hepc1. The 2k upfront position of this gene is almost demethylated (activated) in the Flow through regime whereas the opposite is the case in the RAS regime. The hepc1 (hepcidin-1) molecule is a major player in iron homeostasis and may also bestow antimicrobial activity due to its former property. Hence, in one embodiment the method comprises testing the methylation status of the hepcidin-1 gene, and e.g. use this as a quality predictor.

Table A below is a ranked list of top 20 out of 1000 totally selected genes using window of 2000 bp upstream region for each predicted gene. The gene list is ranked by absolute differences (ratio minus ratio) comparing two different production regimes: flow-through and RAS regime. The ratio value is calculated from the number of methylated CpG sites divided by the total number of CpG sites per feature. Both regimes were standardized in terms of genetic background (same broodstock) and both were smolt matured up to the smolt window and comprised smolt of same size. Columns listed from left to right: gene, number of CpG sites of this gene of the RAS regime, number of methylated CpG sites of this gene at the RAS regime, number of CpG sites of this gene of the Flow through (FT) regime, number of methylated CpG sites of this gene of the FT regime, ratio of methylation of this gene of the RAS regime, ratio of methylation of this gene of the FT regime.

TABLE A

| | | RAS | | FT- | | |
|---|---|---|---|---|---|---|
| | RAS | sites, | FT- | sites, | RAS | FT- |
| Feature/gene | sites | met | sites | met | ratio | ratio |
| LOC106578259 | 24 | 1 | 13 | 13 | 0.04 | 1 |
| i2c2 | 26 | 24 | 13 | 0 | 0.92 | 0 |
| hepc1 | 28 | 27 | 22 | 1 | 0.96 | 0.05 |
| LOC106561688 | 41 | 0 | 64 | 58 | 0 | 0.91 |
| LOC106603928 | 16 | 0 | 10 | 9 | 0 | 0.90 |
| LOC106582077 | 34 | 0 | 10 | 9 | 0 | 0.90 |
| LOC106604853 | 129 | 119 | 41 | 1 | 0.92 | 0.02 |
| LOC106572469 | 26 | 25 | 22 | 2 | 0.96 | 0.09 |
| LOC106588302 | 54 | 4 | 36 | 34 | 0.07 | 0.94 |
| LOC106571646 | 49 | 46 | 14 | 1 | 0.94 | 0.07 |
| LOC106601362 | 13 | 12 | 52 | 3 | 0.92 | 0.06 |
| LOC106584016 | 114 | 16 | 11 | 11 | 0.14 | 1 |
| LOC106611981 | 29 | 2 | 14 | 13 | 0.07 | 0.93 |
| LOC106589905 | 24 | 23 | 10 | 1 | 0.96 | 0.10 |
| LOC106564914 | 73 | 67 | 43 | 3 | 0.92 | 0.07 |
| LOC106565121 | 70 | 59 | 11 | 0 | 0.84 | 0 |
| LOC100380863 | 14 | 1 | 32 | 29 | 0.07 | 0.91 |
| LOC 106600693 | 217 | 36 | 14 | 14 | 0.17 | 1 |
| kiaa1211 | 89 | 74 | 13 | 0 | 0.83 | 0 |

The findings from the analysis reported shows that epigenetic signatures can be used as authenticators, e.g. for traceability of fish.

Hence, in one embodiment the method comprises the steps of comparing epigenetic signatures, and preferably additionally also gene expression profiles, from different fish production regimes (seed or smolt or grow out phase) and accompanying environments, and/or comparing such signatures and profiles with performance data, and/or comparing such signatures and expression profiles with production protocols, and/or comparing such signatures and profiles with accumulated databanks of signatures, protocols and performance data.

As a result of such method steps, one would be able e.g. to one or more of:
Distinguish between different production regimes with accompanying protocols and records and corresponding sea phase performance and product qualities;
Provide feedback to producers for optimization of regimes and protocols;
Verify quality and origin of seeds/smolt and farmed fish and resulting products, and hence e.g. assist in building and protecting brands, or to determine origin of escapees.

In one embodiment, the use of such method makes it possible to determine the quality of the fish, such as to distinguish between different fish quality, such as for smolt, farmed fish, such as in predicting the resulting sea phase performance, without assessing single quality parameters, or fish characteristics. In one preferred embodiment the method is for use to verify smolt status, and/or to optimize smolt production quality.

In the method of the invention, the epigenetic signatures are hence used as a management tool and/or as means for objective biological documentation e.g. for quality. The method may hence form part of a bioproduction. Further, the invention provides a concept using the methylome to provide procedures for both verifying and optimizing the smolt and other seed production and fish farming and the quality of such. The quality obtained is at least in accordance with the requirements of the Norwegian regulations in “The animal welfare act”, “The Food act” and “The aqua culture act”.

The biological/genetics/epigenetics basis of how to achieve these results is based on the knowledge that the epigenome, transcriptome and associated processes provide information about what is going on in the individual at various developmental phases and when exposed to various regimes. This knowledge is accompanied with the employment of the best contemporary technologies available to reveal the natures secrets. Advanced sequencing, translation/bioinformatics and multivariate statistical methods are employed to develop and present relevant profiles and combine and compare them with regime protocols and performance.

For the analysis and comparison/correlation steps, steps iii) and iv) the obtained epigenetic signatures of the sample are analysed and compared with existing data. This may include correlation analysis between either of several epigenetic signatures; the epigenetic signatures and fish performance, robustness or health; or of the epigenetic signatures and production protocols. For the analysis of the epigenetic signatures/epigenome this may include identification of methylation variations and differentially methylated regions, such as of hypomethylation or hypermethylation. The method hence comprises steps to reveal and identify DNA methylation patterns. The epigenetic signatures obtained, may hence give information of the methylation pattern of regulatory parts of the genome as well as of coding gene regions. The steps preferably comprise the comparing of methylation profiles and the identification of methylation variations. Scatter plots may be generated, which together with adequate bioinformatics and statistics tools will suggest or identify correlations and relationships between the variables, e.g. between different epigenetic signatures, or between a given epigenetic signature and the performance data of the bank from earlier collected samples.

Although there is restricted knowledge about the dynamics of the epigenetic genomic anatomy of fishes, a main part of which is the methylation pattern changes along with development and bio rhythms, a general feature of vertebrates may apply also to most fishes: parental methylation are heavily stripped off during gametogenesis and early embryonic development but also at different modus: first in the male pronucleus as an active demethylation process and later in both parental chromosomes as a passive process during replication and cell divisions. Those parental methylations that overcome the mentioned gametogenic and embryonic reprogramming will represent transgenerational epigenetic inheritance. In addition, a considerable number of genes of vertebrates are differentially expressed in the offspring related to the parent of origin: a copy (allele) of a specific gene inherited from one parent may be expressed whereas the other allele of the same gene inherited from the other parent may be non-expressed. This parent of origin specific expression is called genomic imprinting and is bestowed by parent-of origin differential methylation. During embryonic phase and later on in development, into adult life and aging, there is an initial re-methylation of CpG sites and mostly none of the CpG islands, followed by both global and organ and gene specific methylation and demethylation for the purpose to take care of normal differentiation and development. The diverse tool package available for methylation and demethylation mechanisms (CpG sites, CpG islands, gene bodies, gene promoters, enhancers etc.) makes the methylome a major instrument in maturation, biorhythms, handling disease and recovery and aging, and in epigenetic responses to environmental effectors and managemental regimes. The rationale behind this is that a series of genes have to come into play and interplay at different phases and influences (either on/off or quantity) whereas most housekeeping genes are on duty on a continuous basis. In parallel with the reprogramming, there is methylome memory established which carry on the whole lifespan of an individual, as explained below as well as transgenerational inheritance as explained above.

There are differential gene expressions depending on the degree of maturation, age and environment. Genes are differentially expressed during an individual's maturation and aging and this again is governed by endogenous clocks (development, differentiation, biorhythms) and exogenous influences, all of which are released through the main regulator, which is the epigenome, with the methylome as a major contributor.

Hence, there is a «biological clock» and a status of maturation reflected through the degree of both global as well as tissue/organ and gene specific methylation, demethylation and expression profiles. In one embodiment of the invention, the degree of gene methylation is used in determining the smolt status, the degree of maturation or smolt quality. Further, in one embodiment, the method is for use in determination of the biological age of farmed fish, or further to determine correlations between biological age and chronological age. Hence, in addition to physiological and behavioural characteristics, the identified fish characteristics may comprise determination of age.

Accordingly, candidate genes can be identified playing a putative crucial role at certain life developmental phases or at certain tissue/organ differentiation/specialization phases in addition to the housekeeping genes running on a more continuous basis. Also, these phases and rhythms can be synchronized and accelerated with environmental manipulation like with photo and temperature programs, respectively, e.g. in smolt production. Hence, as part of the sequencing and analysing steps of the method, candidate genes are selected and the epigenetic signatures of these are obtained. These may be compared to methylation information of the databank for the same genes, i.e. the analyses comprise comparing the obtained methylation information of candidate genes with the respective information of the databank for the same genes, e.g. comparing the methylation profiles and identify methylation variations. The methylome or the methylome signature can be studied both at specific points in time (real time) or as a memory signature identifying an experienced regime or environmental impact, which could be both good or inferior.

The methylome has a memory although there is also a continuous reprogramming going on along with the development. This methylome memory pattern or signature can consequently be employed as a reflector of the environmental influences the individual has experienced during its development. Hence, the methylation status of candidate genes will add to both verifying quality as well as to provide more precise feedback to production regimes. Transcripts of such genes or a global transcriptome will add information to the methylome profiles, the former being restricted to time window expression profiles and quantitation without any memory, whereas the latter reflects the regulator landscape and can be memorized, programmed, reprogrammed and inherited.

An individual has its own endogenous biological clock, e.g. for development and aging, and rhythm, e.g. for chronobiology; year, season, lunar, day, night. This is for the major part driven by the methylome, as the methyl groups act as brakes and accelerators and corresponding regulators like hormones. This again can be triggered, accelerated or synchronized, by environment and production regimes, such as e.g. temperature, photoperiod regimes (day and night length etc.). This implies that epigenetic programming can be achieved and managed through environmental stimuli and factors, as an epigenetic exogenous programming. This again means that the method steps of the invention comprising the provision of epigenetic profiling analysis (steps i-iii) along with the correlation steps (iv) wherein the obtained data is e.g. compared with performance data and production regimes, can further potentially include corresponding protocol alterations. This can be employed as a husbandry tool to produce the best possible fish for its purpose. In a preferred embodiment, the method is used to provide the most robust smolt, in optimizing the smolt production, in the preparation of quality smolt, such that in producing high yields and healthy smolt.

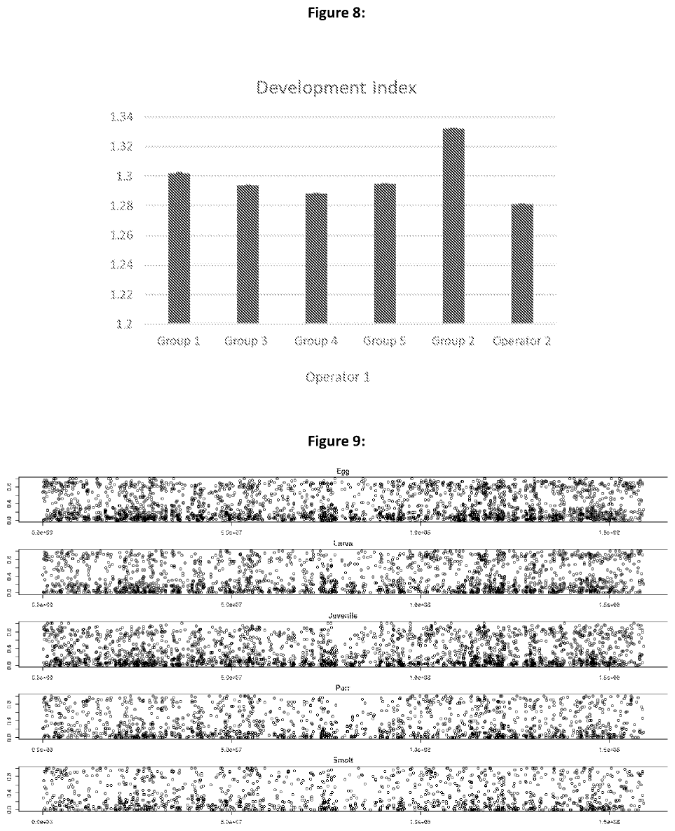

Hence, the epigenetic signatures obtained may be used as determinator of fish or smolt maturation, as an “epigenetic clock”. Further, the epigenetic signatures and method of the invention may provide a development status at a certain life phase (Development index). For instance, when comparing groups of smolt from the same operator as well as smolts between operators with different production regimes, the applicant has found that one is able to reveal contrasting difference in methylation frequency, i.e. number of CpG sites methylated compared to CpG sites present in the genome. This methylation frequency parameter may be a useful reflector of maturation since it reflects that different number of genes are in action in the two systems. Hence, given that there is a correlation between number of activated genes and methylation frequency, and this again is correlated to level of maturity and development, the one regime with the lowest frequency of methylation is the one with the most mature fish. Strong evidence for the above being the case came out when blindly analysing 5 groups of smolt within one operator. One group came out with particularly high development index (Group 2), i.e. the inverse ratio of methylation frequency, compared to the others. After testing, the operator informed that the high-level group (Group 2) was given an extra photoperiod treatment during smoltification. The results are shown in FIG. 7. Hence, this general development index can be regarded as a particularly photoperiod sensitive index and applied as a tool to monitor photoperiod regimes in terms of various structures (protocols) and the effect and outcome of such. Again, this can be used as a tool to optimize photoperiod treatments. Moreover, the development index of groups of smolt from two different operators were analysed. When comparing the two different operators, all groups of the one operator (Operator 1) came out significantly higher than the other one in terms of development index. The results are shown in FIG. 8. Historically operator 1 providing the highest index has nation leading performance in the sea grow out phase in terms of growth rate and robustness. This shows that the provision of epigenetic signatures, and particularly the methylation frequency, may be used in the provision of a development status at a certain life phase. A potential test for methylation level of hepcidin-1 and other gene candidates displaying high methylation contrasts between compared regimes in our presented data set (Table A) as well as residing in our available database is expected to add power to the general development index as depicted in FIGS. 7 and 8. In one embodiment, the method comprises analysing the methylation level of hepcidin-1.

Further, in one embodiment of the invention, a timeline biological clock based on epigenetic (i.e. methyl) signatures is provided for the following salmon development steps: fertilized egg, yolk sack larvae, fry, parr and smolt. This may be used as a Salmon methyl clock, a “SalmoClock”.

The signatures are displayed in several dimensions or by the following features and distribution to allow for maximum informativeness in revealing uniqueness and contrasts between the listed steps of the life phases: chromosome distribution, methyl islands, transcription sites, gene body and 2k upstream gene. Strong contrasts and unique features were revealed for each step both in terms of patterns but also in terms of phase specific genes.

The strongest contrasts between life phases are seen between yolk sack larvae and fry, as start feeding changes a lot in digestion, metabolisms etc. and between parr and smolt. The “SalmoClock” will provide a robust guidance for safe development of robust smolt, the latter being one of the most critical challenges in current salmon farming.

The epigenome, in contradiction to the transcriptome which has volatile molecules not leaving any trace for memory, has a memory, as described above, and moreover: it can also be inherited through methylome signatures passed to next generation via germ cells. This implies that epigenetics can be deployed to optimize next generation performance through inherited regulatory signals. Also, it implies that epigenetics may be combined with breeding to potentially enjoy a new and untapped synergy. In one embodiment, the method of the invention is combined with breeding, e.g. in epigenetics guided fish rearing. For instance, the fish individuals selected for breeding are picked based on their epigenome, not just their genetic value. In one embodiment of the invention, the analysed epigenetic signature of a fish sample is assessed for its relevance for breeding. Also, fish selected for specific traits and markets could be further finetuned to optimize their performance if epigenetics guided rearing of the smolt and food fish could take place on the top of the current breeding. In one embodiment, the prepared epigenetic signatures form part of an epigenetic signature based test system for breeding regimes.

In the bioproduction context, and in a preferred embodiment of the invention, the methylome signatures, transcriptome or expression profiles (of step iii) could thus be exploited as any one or more of the following:

1) A global dynamic methylation pattern (methylation and demethylation) graph (curve) as a function of development (maturation, differentiation and aging) and thus reflecting an individual's relative maturation stage or biological age is established. I.e.: providing the correlation (step iv) between methylation dynamics and maturation/differentiation and aging.

2) A tissue or organ specific dynamic methylation graph reflecting differentiation or maturation is established, to reflect maturation stage and age.

3) As for 1) and 2) differentially methylation and/or methylation reprogramming is correlated to maturation/differentiation and aging.

4) As for 1) and 2) differential gene expression (transcripts and transcriptomes) is correlated to differentiation and aging.

Further, the method may include steps wherein any of the results from the correlations steps above are used as maturation and biological age verifiers and as feedback to production and protocols. Main protocols to optimize in smolt production are light (photoperiods) and temperature regimes. Inferior maturity in smolt production may therefore mostly be related to the structuring of these parameters. In one embodiment, the obtained epigenetic signatures and/or gene expression profiles can be linked with the sea phase performance, e.g. if this is good or inferior.

Along with the accumulation of data for the epigenetic signature data bank and the performance data bank, the method of the invention and value and usefulness of this, will gain strength. Hence, along with the accumulation of global or organ/tissue/cell or gene based epigenetic signatures and expression profiles linked with life phase, and correlated with production regimes/protocols and grow out sea phase performance data, and statistical association calculations between such, the method, both for verification and feedback, will gain strength. In one embodiment, the method requires big data compilation and eventually also likely machine learning (ML) implementation.

Hence, the method may include the use of tools and methods to handle informatics, bioinformatics, statistics or mathematics, which may comprise any one or more of the following, but not being restricted to:

Image and pattern analysis and recognition (e.g. scatter plots), cluster analysis, various comparison and probability biostatistics within regression analysis (least squares, linear and non-linear), multivariate analysis and data dimensional reduction techniques, fish index calculations based on signatures, computer graphics, big and large scale data, machine learning and artificial intelligence techniques to handle complex and vast data together with adequate algorithm and computer software development and/or customization.

Genome location: The 2k upstream of genes is a key genome location of methyl signatures with powerful universal informative value. Analyses of genome wide methylation levels, restricted to those 2k upstream of gene reading frames, display variability far above all other genome location or features when comparing salmon life phases from fertilized egg to smolt as well as comparing different production regimes at smolt window phase. This finding implies that this 2k upstream “methyl universe” is the most robust source from which to find informative methyl patterns as well as concrete gene specific methylation levels linked to, but not restricted to, origin (authentication and traceability) as well as to maturation (unwanted maturation included like sex maturation in the grow out phase), development status, robustness etc.

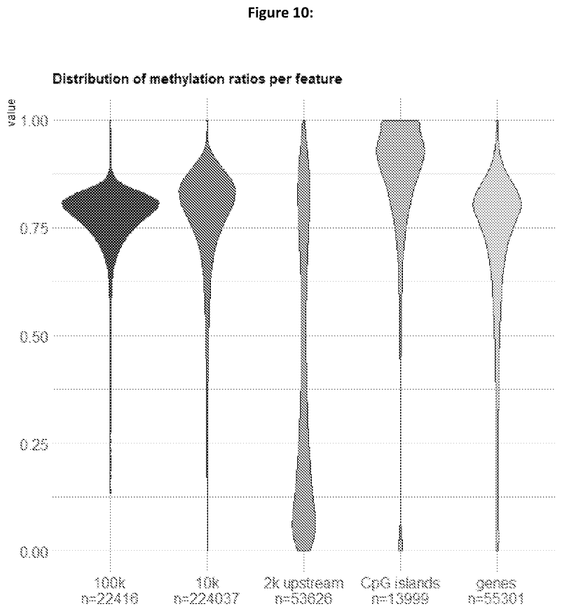

The FIG. 9 displays methylation patterns in five life phases of salmon, displaying chromosome 1 wide methylation level variation 2 k upstream genes of salmon when comparing life phase from egg to smolt. The variation is much less outspoken when displaying it with gene body, or methyl island, or the Chr. 1 as a whole as shown in the violin plot provided in FIG. 10. This figure demonstrates the distribution of methylation values for each feature. Note the 2000 bp upstream region containing transcription start sites differ dramatically in its distribution profile, suggesting this data track offers the most representative resolution of dynamic methylation signals. As one might expect CpG islands tend to be methylated and regions containing transcription start sites (2k) tend to accessible and open relatively to the other data tracks. The relatively scarce methylation plot of the island window (FIG. 10) reflects the expected much lower number of CpG islands compared to the number of CpGs as a whole, which again confirms the robustness and reliability of the method.

Table B below, including four sub tables B1 to B4, provides the top 20 differentially methylated genes comparing five life phases of salmon. Hence, this is a ranked list of top 20 selected genes using window of 2000 bp upstream region for each predicted gene. The gene list is ranked by absolute differences (ratio minus ratio) comparing pairwise life phases of salmon listed from top to bottom of the Table: egg vs larvae, larvae vs juvenile, juvenile vs parr and parr vs smolt. The ratio value is calculated from the number of methylated CpG sites divided by the total number of CpG sites per feature. Columns listed from left to right: gene, number of CpG sites of this gene of a specific life phase, number of methylated CpG sites of this gene of the same life phase, number of CpG sites of this gene of the compared life phase, number of methylated CpG sites of the compared life phase, ratio of methylation of this gene of the first life phase, ratio of methylation of this gene of the compared life phase.

TABLE B
B1 Egg vs Larva:

| | | | | Lar- | | |
|---|---|---|---|---|---|---|
| | | Egg, | Lar- | va, | | |
| Gene | Egg | met | va | met | Egg ratio | Larva ratio |
| LOC106613732 | 83 | 2 | 11 | 11 | 0 | 1.00000000 |
| cherp | 71 | 2 | 13 | 13 | 0.02409639 | 1.00000000 |
| LOC106574000 | 10 | 10 | 16 | 16 | 0.02816901 | 1.00000000 |
| LOC106608775 | 14 | 14 | 88 | 3 | 1.00000000 | 0.03409091 |
| lyric | 65 | 1 | 20 | 1 | 1.00000000 | 0.05000000 |
| LOC106573542 | 126 | 8 | 22 | 21 | 0.01538462 | 0.95454545 |
| LOC106600447 | 94 | 88 | 10 | 10 | 0.06349206 | 1.00000000 |
| LOC106563834 | 10 | 0 | 12 | 0 | 0.93617021 | 0.00000000 |
| LOC106566816 | 28 | 2 | 29 | 27 | 0.00000000 | 0.93103448 |
| LOC106602351 | 47 | 43 | 18 | 18 | 0.07142857 | 1.00000000 |
| LOC106601789 | 58 | 5 | 10 | 0 | 0.91489362 | 0.00000000 |
| daam2 | 12 | 12 | 15 | 15 | 0.08620690 | 1.00000000 |
| LOC106570740 | 45 | 0 | 132 | 12 | 1.00000000 | 0.09090909 |
| LOC106570715 | 68 | 65 | 10 | 9 | 0.00000000 | 0.90000000 |
| LOC106586004 | 31 | 2 | 31 | 2 | 0.95588235 | 0.06451613 |
| LOC106610112 | 15 | 13 | 18 | 17 | 0.06451613 | 0.94444444 |
| LOC106560390 | 41 | 35 | 10 | 0 | 0.86666667 | 0.00000000 |
| LOC106576073 | 43 | 3 | 18 | 0 | 0.85365854 | 0.00000000 |
| LOC106587580 | 57 | 51 | 13 | 12 | 0.06976744 | 0.05263158 |

B2 Larva vs Juvenile

| | | | | | | Juvenile |
|---|---|---|---|---|---|---|
| Gene | Larva | Larva, met | Juvenile | Juvenile, met | Larva ratio | ratio |
| LOC106609100 | 24 | 0 | 34 | 34 | 0.00000000 | 1.00000000 |
| LOC106583289 | 15 | 0 | 14 | 14 | 0.00000000 | 1.00000000 |
| LOC106586831 | 16 | 16 | 28 | 0 | 1.00000000 | 0.00000000 |
| LOC106572469 | 15 | 15 | 50 | 1 | 1.00000000 | 0.02000000 |
| LOC106593522 | 25 | 24 | 12 | 0 | 0.96000000 | 0.00000000 |
| LOC106602974 | 34 | 0 | 23 | 22 | 0.00000000 | 0.95652174 |
| LOC106574163 | 10 | 10 | 16 | 1 | 1.00000000 | 0.06250000 |
| cenph | 13 | 13 | 32 | 2 | 1.00000000 | 0.06250000 |
| LOC106599349 | 10 | 10 | 29 | 2 | 1.00000000 | 0.06896552 |
| LOC106573542 | 10 | 10 | 68 | 5 | 1.00000000 | 0.07352941 |
| LOC106600414 | 13 | 12 | 57 | 0 | 0.92307692 | 0.00000000 |
| LOC106566062 | 13 | 12 | 14 | 0 | 0.92307692 | 0.00000000 |
| LOC106586574 | 11 | 11 | 50 | 4 | 1.00000000 | 0.08000000 |
| LOC106585280 | 12 | 11 | 36 | 0 | 0.91666667 | 0.00000000 |
| LOC106607445 | 11 | 10 | 22 | 0 | 0.90909091 | 0.00000000 |
| LOC106607429 | 44 | 2 | 21 | 20 | 0.04545455 | 0.95238095 |
| LOC106575015 | 13 | 13 | 61 | 6 | 1.00000000 | 0.09836066 |
| LOC106574436 | 10 | 9 | 22 | 0 | 0.90000000 | 0.00000000 |
| LOC106565191 | 12 | 11 | 110 | 2 | 0.91666667 | 0.01818182 |
| LOC106582603 | 57 | 51 | 53 | 0 | 0.89473684 | 0.00000000 |

B3 Juvenile vs Parr:

| | | | | | Juvenile | |
|---|---|---|---|---|---|---|
| Gene | Juvenile | Juvenile, met | Parr | Parr, met | ratio | Parr ratio |
| LOC106607635 | 17 | 0 | 10 | 10 | 0.00000000 | 1.00000000 |
| LOC106565671 | 20 | 0 | 15 | 15 | 0.00000000 | 1.00000000 |
| LOC106568484 | 11 | 11 | 26 | 1 | 1.00000000 | 0.03846154 |
| LOC106602974 | 23 | 22 | 27 | 0 | 0.95652174 | 0.00000000 |
| LOC106566615 | 43 | 2 | 12 | 12 | 0.04651163 | 1.00000000 |
| LOC106607429 | 21 | 20 | 41 | 0 | 0.95238095 | 0.00000000 |
| LOC106579413 | 19 | 18 | 12 | 0 | 0.94736842 | 0.00000000 |
| LOC106578867 | 49 | 3 | 15 | 15 | 0.06122449 | 1.00000000 |
| LOC106590324 | 30 | 29 | 34 | 1 | 0.96666667 | 0.02941176 |
| henmt1 | 42 | 3 | 11 | 11 | 0.07142857 | 1.00000000 |
| LOC106573916 | 11 | 11 | 14 | 1 | 1.00000000 | 0.07142857 |
| LOC106588755 | 21 | 0 | 14 | 13 | 0.00000000 | 0.92857143 |
| LOC106563969 | 13 | 12 | 16 | 0 | 0.92307692 | 0.00000000 |
| LOC106586458 | 25 | 23 | 16 | 0 | 0.92000000 | 0.00000000 |
| LOC106602488 | 48 | 4 | 13 | 13 | 0.08333333 | 1.00000000 |
| LOC106609100 | 34 | 34 | 147 | 14 | 1.00000000 | 0.09523810 |
| LOC106566062 | 14 | 0 | 21 | 19 | 0.00000000 | 0.90476190 |
| LOC106601375 | 10 | 9 | 10 | 0 | 0.90000000 | 0.00000000 |
| LOC106601757 | 10 | 9 | 13 | 0 | 0.90000000 | 0.00000000 |
| LOC106560344 | 38 | 0 | 10 | 9 | 0.00000000 | 0.90000000 |

B4 Parr vs smolt

| | | Parr, | | Smolt, | | |
|---|---|---|---|---|---|---|
| Gene | Parr | met | Smolt | met | Parr ratio | Smolt, ratio |
| LOC106606477 | 12 | 12 | 29 | 0 | 1.00000000 | 0.00000000 |
| LOC106608475 | 19 | 19 | 16 | 0 | 1.00000000 | 0.00000000 |
| LOC106609239 | 25 | 0 | 13 | 13 | 0.00000000 | 1.00000000 |
| LOC106609432 | 10 | 0 | 19 | 19 | 0.00000000 | 1.00000000 |
| LOC106562856 | 10 | 10 | 18 | 0 | 1.00000000 | 0.00000000 |
| LOC106566615 | 12 | 12 | 26 | 0 | 1.00000000 | 0.00000000 |
| LOC106567585 | 23 | 0 | 17 | 17 | 0.00000000 | 1.00000000 |
| LOC106578738 | 11 | 11 | 30 | 0 | 1.00000000 | 0.00000000 |
| ptprc | 24 | 23 | 15 | 0 | 0.95833333 | 0.00000000 |
| LOC106603088 | 108 | 6 | 11 | 11 | 0.05555556 | 1.00000000 |
| LOC106607762 | 10 | 0 | 16 | 15 | 0.00000000 | 0.93750000 |
| gemin6 | 23 | 1 | 37 | 36 | 0.04347826 | 0.97297297 |
| LOC106567215 | 14 | 1 | 10 | 10 | 0.07142857 | 1.00000000 |
| LOC106580999 | 11 | 0 | 13 | 12 | 0.00000000 | 0.92307692 |
| LOC106566713 | 23 | 22 | 53 | 2 | 0.95652174 | 0.03773585 |
| LOC106599448 | 10 | 0 | 12 | 11 | 0.00000000 | 0.91666667 |
| LOC106573991 | 12 | 1 | 10 | 10 | 0.08333333 | 1.00000000 |
| LOC106588411 | 12 | 11 | 21 | 0 | 0.91666667 | 0.00000000 |
| LOC106602908 | 11 | 10 | 25 | 0 | 0.90909091 | 0.00000000 |
| LOC106589831 | 32 | 29 | 11 | 0 | 0.90625000 | 0.00000000 |

Some typical life phases, organs/tissues, processes and corresponding candidate genes to pursue are e.g. any one or more of the following:

Embryonic phase: Homeobox gene family, e.g. Hypoxia Inducible Factor (HIF).

Hatching phase: HIF and heat and cold shock protein genes, e.g. Cold Inducible RNA-binding protein, CIRBP.

Yolk sac larva phase: HIF and heat and cold shock protein genes e.g. CIRP, Genes involved in central nervous system development, hypothalamus, pituitary gland, olfactory bulb.

Fry (start feeding) phase: Genes involved in further development of said organ, and also of the liver, kidney, guts, gills. Genes involved in regulating releasing hormones from hypothalamus and hormones from pituitary gland: Growth hormone (GH), genes involved in other metabolic pathways and in digestion.

On-growing until photoperiod phase: GH, thyroxine, corticoids, other genes involved with hyperosmotic like prolactin («the fresh water hormone), Na-K-ATPase, carbonic acid anhydrase, other genes involved with metabolic pathways and digestion, genes involved in sensing fragrance in the water with impact on behaviour and feeding etc.

Photoperiod, i.e. the phase preparing for coping with salt tolerance and other stressors related to transfer to sea through metamorphosis: genes activated by photoperiod regime and dark triggered hormone like melatonin, genes involved with hypoosmotic regulation, glycogenolysis and lipidolysis: (more slim and less fat body), genes involved in increased purine retention in cutis (silver shiny), (NA-K-ATPase, Thyroxine, corticosteroids (increased protein catabolism, hyperglycemia etc). While prolactin is the freshwater hormone, mineral corticoids, e.g. cortisol, is the saltwater hormone. Oxygen transporting genes (haemoglobins), genes involved in sensing.

Smolt window. Status of genes involved with the development of the chloride cells in the gills, e.g. growth hormone (GH), status of salt tolerance genes like NA-K-ATPase, thyroxine, mineral corticoids, ion excretion from gills, water reabsorption from guts and kidney, stress related hormones (corticoids), catecholamines, insulin, immune defence genes like major histocompatibility complex (MHC) class I and II, T-cell receptor, toll-like receptor. Genes involved in behaviour like those triggering smolt to swim with the flow contrary to the parr swimming against the flow.

Genes differentially methylated comparing RAS vs Flow through (FT): i2c2: Eukaryotic translation initiation factor 2C 2. Involved in protein biosynthesis and RNA-mediated gene silencing.

hepc1: Hepcidin-1 The hepcl (hepcidin-1) molecule is a major player in iron homeostasis and may also bestow antimicrobial activity due to its former property.

Genes differentially methylated across salmon life phases: “SalmoClock”:

Egg—Larva:

cherp: Calcium homeostasis endoplasmic reticulum protein. Involved in cell calcium ion homeostasis.

lyric: The amino acid sequence of 3D3/lyric indicates that it may be a type-1b membrane protein with a single transmembrane domain (TMD). Involved with protein kinase B signaling, autophagy and angiogenesis.

daam2: Disheveled associated activator of morphogenesis. Important development gene. Key regulator of the Wnt signaling pathway, which is required for various developmental processes e.g.: dorsal patterning, left/right symmetry, myelination of spinal cord. Together with DAAM1, required for myocardial maturation.

LARVA—JUVENILE:

cenph: Component of the CENPA-NAC (nucleosome-associated) complex, a complex that plays a central role in assembly of kinetochore proteins, mitotic progression and chromosome segregation.

JUVENILE—PARR:

henmt1: A methyltransferase that adds a 2′-O-methyl group at the 3′-end of piRNAs, a class of 24 to 30 nucleotide RNAs that are generated by a Dicer-independent mechanism and are primarily derived from transposons and other repeated sequence elements. This probably protects the 3′-end of piRNAs from uridylation activity and subsequent degradation. Stabilization of piRNAs is essential for gametogenesis.

PARR vs SMOLT:

ptprc: Protein tyrosine-protein phosphatase receptor type C. Required for T-cell activation through the antigen receptor. Acts as a positive regulator of T-cell coactivation upon binding to DPP4. A CD 45 antigen involved with stem cell development and leucocyte differentiation.

gemin6: The SMN complex plays a catalyst role in the assembly of small nuclear ribonucleoproteins (snRNPs), the building blocks of the spliceosome. Thereby, plays an important role in the splicing of cellular pre-mRNAs.

In one embodiment, the method comprises that any one or more of the above-mentioned candidate genes or groups of genes are selected and the epigenetic signatures, the methylation status, and optionally also expression profiles of these are obtained. In one embodiment, the method comprises preparing at least one epigenetic signature for one or more of the genes from the group of i2c2, hepc1, cherp, lyric, daam2, cenph, henmt1, ptprc and gemin6.

Further, in one embodiment of the invention, the prepared epigenetic signatures, particularly for any such candidate genes, form part of an epigenetic signature based test system for any one or more of the following, but not restricted to, qualitie; robustness, maturation, biological age, authentification (traceability), or in breeding regimes, of bony fish.

Based on the findings (observations) from the identified epigenetic signatures and optional gene expression profiles (steps iii), and from the results of the correlations steps (steps iv) of the method, adequate measures, i.e. additional potential steps of the method, that can be taken are indicated below:

Observation: The general (global) demethylation and/or differential methylome and transcriptome is not satisfactory advanced compared to life phase, i.e. is immature.

Measure; Implement extended photoperiod or optimized day and night regime and leave more time.

Observation: Tissue/organ specific differentiation is not satisfactory developed, including chloride cells.

Measure: Extend or re-structure photoperiod with e.g. strengthen “winter modus” (see paragraph below) and/or extend time for maturation with accompanying lower temperature.

Observation: Gene specific methylome and gene specific expression profile related to smoltification is not in place. I.e. «the freshwater hormone» prolactin should be downregulated, thyroxine, NA-K-ATPase and mineral corticoids, i.e. cortisol («the salt-water hormone») should be upregulated, O₂-sensitive haemoglobin variants should be upregulated due to preparing for lower oxygen tension in seawater etc.

Measure: Change day/night ratio to initially have shorter days (winter period) before extending day period to synchronize the various preparing processes in the fish. Consider exposing the smolt to more salinity and lower O₂-tension for a defined period to stimulate chloride cell development and haemoglobin variant switch.

Observation: Sub-optimal performance in the grow out sea phase and post-harvest qualities.

Measure: Compare through advanced statistics the performance data with smolt production regime/protocols and with smolt methylome signatures and expression profiles. Depending on type of sea phase malperformance (early, mid phase or late death or sickness, cause, carcass qualities) and correlation analysis results with profiles and smolt regimes/protocols: sort out targeted feedback to alter production protocols to the better. For instance, if the grow out records show inferior survival rate due to infections and the epigenetic signatures and expression profiles of the corresponding smolt reflects inferior maturation and differentiation of immune organs and tissues, the feedback to hatchery protocols should be to leave more time for the fry to mature and/or to optimize feed formula.

A series of parameters can be adjusted depending on if the plant is a flow through system or a recirculation aquaculture system (RAS). The major common parameters are:

Photoperiod regime (day/night ratio) and time, temperature and time. In addition to these, both regimes can alter: Feed and feeding regimes, including parameters as water flow, fish density, oxygen and CO₂ concentration, salinity exposure, handling regimes (e.g. moving fish to new compartments along with growth).

Hence, in one embodiment of the method, fish farming production regimes and corresponding protocols are amended, such as optimized, based on feedback from the prepared epigenetic signatures linked with the performance data.

Examples of appropriate procedures, tools and instruments to use in the method are provided herein. For the sampling and sequencing steps, analytical methods are to be used, e.g. including DNA isolation from relevant samples, e.g. from smolt organs and tissues. Genomic DNA is isolated from relevant organs, e.g. from Atlantic salmon (Salmo salar) using standard, well known protocols. E.g. DNA is isolated using the kit “DNeasy Blood and tissue” kit (Qiagen) following the recommended protocol. Shortly: A suitable amount of biological material from the fish tissue or organ is digested by proteinase K and corresponding buffer. The solution is mixed with relevant kit buffer and ethanol and centrifuged through a spin column where the DNA is bound to a filter with affinity for this. Appropriate kit buffers are used for washing and eluting the DNA from the spin column.

Alternative methods for isolating genomic DNA from the fish samples may be used, e.g. phenol:chloroform may be used to extract proteins and other molecules after the proteinase K treatment. DNA may then be precipitated using ethanol and salt.

The quantity and purity of the DNA may be recorded using e.g. Qubit and Nanodrop instruments, respectively.

DNA sequencing: Genomic DNA is sequenced e.g. using the MinION instrument from Oxford Nanopore Technologies (ONT) and associated sequencing kit: Ligation

Sequencing Kit (SQK-LSK109). The recommended protocol of the producer was followed. Shortly: The genomic DNA is treated with kit ingredients to repair the ends of the molecules as well as generating a 5′ A-overhang. Sequencing adapters, containing the necessary DNA sequence and molecules in order to guide the DNA molecule through the pores in the flow cell membrane of the MinION instrument, are ligated to the genomic DNA fragments. The DNA molecules are then loaded onto the flow cell and the sequencing process runs until a satisfactory number of DNA sequences are produced. Both R9.5.1 Flow cells as well as Flongle flow cells may be used, but the R9.5.1 MinION flow cell has the preferred high capacity when whole genome sequencing is performed. The MinION sequencing instrument from ONT is used. However, other instruments from the same providers (GridION and PromethION) operating with the same DNA sequencing technology as MinION, but with higher throughput capacity, may also be used. The sequencing process is controlled with the accompanied software MINKNOW provided by ONT.

RNA isolation: RNA may be isolated from relevant fish sample materials, such as from smolt organs and tissues.

The epigenetic fingerprint (signatures) obtained, e.g. as described in the Examples, give information of the methylation pattern of regulatory parts of the genome as well of coding gene regions. These methylation patterns affect the expression of genes that have influence on important traits of the fish—both production traits as well as other traits and performance characteristics. This gene expression influence may be in the form of increased or decreased transcription of specific genes. Thus, the amount of mRNA is affected. In some situations, it may be of interest to analyze the relative amount of specific mRNA molecules present in the fish sample material, such as in relevant organs and tissue of fry and smolt as well as adult individuals, and the method may comprise this.

Total RNA is isolated using standard techniques. Samples from relevant organs and tissue from fish is placed on RNA preserving buffer (RNAlater) or immediately frozen in liquid nitrogen. Total RNA and/or mRNA is isolated. Either by spin column methods as Qiagens RNeasy Mini Kit or similar kits from other providers. Also, traditional methods based on Trizol extraction of proteins works perfectly.

RNA sequencing and/or quantification (Nanopore or qPCR, stabilized on RNA Later stabilization solution): Isolated total RNA or mRNA can be sequenced using several sequencing methods. For example, RNA molecules may be sequenced directly using the Oxford Nanopore kit “Direct RNA sequencing kit” and the same instrument as described above, MinION, for direct DNA sequencing. By sequencing the RNA molecule directly, base modifications in the molecule can be detected. The isolated RNA molecules can also be transcribed to cDNA which subsequently are sequenced using either the Nanopore technology or other available techniques and instruments.

In one embodiment, the method comprises a step of transcriptome analysis based e.g. on data from microarray, Nanopore based sequencing or qPCR: Bioinformatics Statistical analysis displaying correlation between methylome signatures, gene expression profiles, production protocols and sea phase performance data, such as contract biostatistics and programmer expertise.

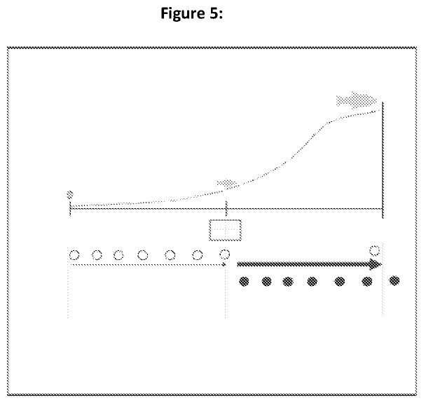

To summarize and illustrate, the main concept of the invention comprises the preparing and comparing of epigenetic signatures, as displayed in Example 1 and above. The epigenetic signatures may be compared with fish production regimes (protocols) and preferably with performance data records (growth rate, health and carcass qualities). Records from fertilized egg to harvest with emphasis on signatures at seed/smolt level and performance data at grow out phase are displayed in FIG. 5. This figure illustrates the concept of the invention comprising sampling of specimens (fish sample material), generating epigenetic signatures and preferably recording performance data. In the smolt phase, left half of FIG. 5, epigenetic signatures (open dots or rings) are generated from a series of samples from several phases of smolt production in the hatchery e.g. fertilized egg, yolk sac larva, hatched fry, on grown fry, parr, photoperiod, smoltification, smolt window included (window symbol) also covering a series of relevant organs. The thin accompanying arrow reflects the production protocol. The right part of the FIG. 5 reflects the sea phase grow out phase with post-harvest included. Performance on a continuous basis may be recorded consisting of, but not limited to, growth rate, survival rate, health records as well as carcass qualities (black dots). Samples may also be taken from several relevant organs/tissues of culled fish as basis for generating epigenetic signature of mature fish (open dot).

Hence, by employing contemporary biostatistics or informatics tools in data mining, compiling and comparison, this enables the provision of either of the following major solutions:
Distinguish between different production regimes, i.e. different production regimes with accompanying protocols, and further distinguishing between sea phase performance and resulting product qualities from the different production regimes.
Provide feedback to optimize production through adequate alteration of protocols guided by epigenetic signatures.
Provide prediction of performance of fish in the sea phase based on smolt epigenetic signature.
Establish epigenetic signature-based tracking of fish to assist in: authenticity, brand building and brand protection of seafood and detection of origin of escaped farmed fish. This will be based on the establishment of signature databases of various producers with which signatures of seafood products in the markets or living escapees will be compared.

The following Examples are provided to illustrate the invention in accordance with the principles of the invention but are not to be construed as limiting the invention in any way.

### EXAMPLES

### Example 1

### Preparation and Analysis of Epigenetic Signatures from Kidney and Liver of Atlantic Salmon

Samples from liver and posterior kidney of 60-70 grams smolt of cultured Atlantic salmon (Salmo salar) approaching the smoltification window were taken. DNA were isolated from the samples employing the following procedure: The samples were stored at −80° C. and thawed on ice before the isolation procedure. A DNeasy® blood and tissue kit (Qiagen, REF: 69504) was used to extract total DNA from the collected samples according to the manufacturer's manual.

The DNA samples from the two selected tissues (kidney and liver) were sequenced using Oxford Nanopore Sequencing in accordance with the following protocol. The DNA samples were measured with nanodrop to ensure the DNA quality matches the DNA sample criteria set by manufacturer. For the sequencing, a MinION and FLO-MIN106 flow cells was used. The sequencing was performed according to the Nanopore protocol (SQK-LSK109), and the MiniKnow software (provided by Oxford Nanopore) carried out the data acquisition and the real-time basecalling. After basecalling with the data processing toolkit containing the Oxford Nanopore basecalling algorithm, GUPPY, the fastq files were merged into one fastq file pr.

tissue.

Table 1 depicts in key figures the outcome from the described sequencing process from the two organs.

TABLE 1
Number of reads, length of reads and total number of bases generated from
DNA isolated and sequenced according to described protocols

| | N reads | Mean read length | Max length | Total bases |
|---|---|---|---|---|
| Kidney | 1 869 349 | 1396.79 | 84486 | 2 611 082 610 |
| Liver | 1 435 985 | 2676.19 | 214566 | 3 842 972 596 |

Generation of global, organ and gene specific methylation profiles:

The reads were then aligned to the Atlantic salmon (Salmo salar) reference genome using Burrows-Weeler (BWA) software package. Methylated CG sites were recovered using the Oxford Nanopore software package, NANOPOLISH, for signal level analysis to detect methyl modifications, here 5-methylcytocine of CG sites of the sequence data. Instances with a statistical log likelihood >|2.5| were considered a valid instance.

As displayed in Table 2 from the 2,9 giga base sized salmon genome approximately 48 million CpG sites were retrieved, all of which are potential methylation candidates. Subsequent to quality filtering data from approximately 14 and 17 million CpG sites of kidney and liver were combined, respectively, approximately 50% of which are residing inside gene bodies.

TABLE 2
Total CpG instances before and after quality filtering
and combined sites total and in gene body.

| | | CpG | | Combined | |
|---|---|---|---|---|---|
| | | instances | | CpG | |
| | CpG | after quality | Combined | sites inside | Mean |
| | instances | filter | CpG sites | genes | coverage |
| Kidney | 44 736 073 | 25 286 671 | 14 271 265 | 6 668 377 | 2.60816 |
| Liver | 53 893 389 | 31 349 771 | 17 239 686 | 8 473 963 | 2.68353 |

Overlapping instances were combined and the number of instances, coverage and degree of methylation (ranges from 0, unmethylated to 1, methylated) were reported for each of the gene boundaries. Using the genome annotation mentioned above the genomic coordinates of the 57 932 predicted genes, 53 715 of which appeared in either of the tissues and 47 436 in both tissues, were extracted. Data was successfully generated from approximately 95% of the genes. A majority of genes were methylated and also a majority displayed a degree of methylation from 0.6 to 1.0 on a scale from 0 to 1.0. Overall, kidney came out with higher value of methylation than liver.

The literature indicates that bony fish (teleost) have more methylated CpG sites than other taxons, that vertebrate intergenic methylation tends to repress gene expression whereas gene body associated methylation may enhance expression if combined with hypo- or unmethylated promoter, although exceptions exist.

Key figures of these findings are presented in Table 3 and FIG. 1 and FIG. 2. FIG. 1 is a Scatter plot of average methylation values per gene and per organ (liver and kidney), with a correlation coefficient r=0.544. The plot displays all genes before reduction from well above 53 000 to 6 583.

FIG. 2 is a frequency distribution graph where genes are sorted by methylation values, wherein methylation values for kidney is dark grey, liver is white and where the global picture, the data of the two tissues merged, is light grey. The graph contains information from all genes before reduction from 53 715 to 6 583. FIG. 2 embraces multiple sets of information: methylation values, frequency distribution of genes by methylation value, as also reflected in Table 3, and the difference between kidney and liver as of methylation value. In addition, FIG. 2 reflects that kidney being overall more methylated than liver.

Both the density distribution of the scatterplot of FIG. 1 as well as the frequency graph depicted in FIG. 2 show that the majority of the genes have a methylation value of >0.6 and <0.9 in both tissues.

TABLE 3
Distribution of methylation values of the genes in kidney and
liver grouped with the following methylation cut offs:
<0.25, >0.25 and <0.5, >0.5 and <0.75, and >0.75.

| | | Methyla- | Methyla- | | |
|---|---|---|---|---|---|
| 53,715 | Methyla- | tion > 0.25 | tion > 0.5 | Methyla- | Missing |
| genes | tion < 0.25 | and < 0.5 | and < 0.75 | tion > 0.75 | data |
| Kidney | 2675 | 2910 | 12 799 | 29 977 | 3298 |
| Liver | 2922 | 3631 | 16 036 | 25 546 | 3664 |

Reduction of the Number of Genes:

After the generation of the methylation profiles a reduction of the number of genes to those with official gene symbol and with data from both tissues was performed. Genes by tissue, by large difference in methylation, as well as highlighting genes specialized in development and differentiation, are reported.

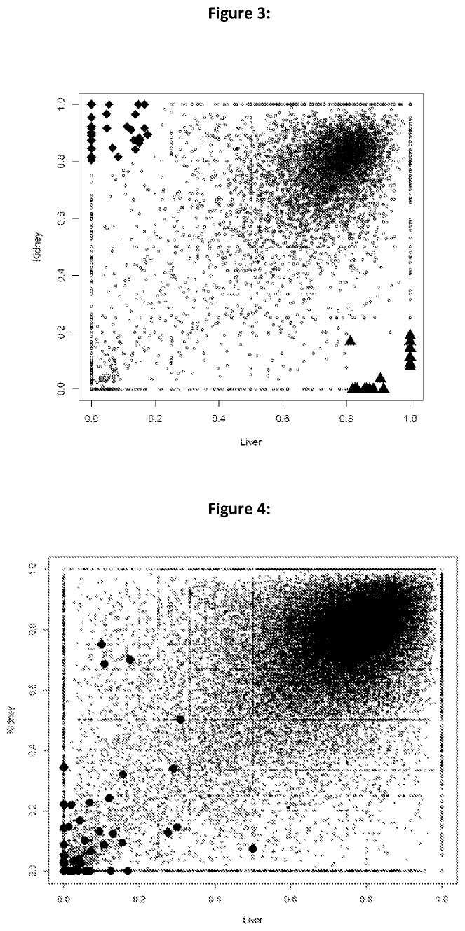

Hence, the number of genes was reduced by including only genes with an official gene symbol, and data in both tissues (liver and kidney). This resulted in 6583 genes. Further, genes with a “large” difference in methylation were selected, using an arbitrary cut off selected to <0.2 AND >0.8 in both tissues. Genes have been included which are unmethylated in both tissues (==0). The results are found in Table 4 (methylated in liver and not in kidney), Table 5 (methylated in kidney and not in liver) and Table 6 (unmethylated in both tissues), as well as in FIG. 3.

FIG. 3 hence provides a scatter plot of selected genes with a “large” difference in methylation value together with the global picture, and including 6583 genes, using an arbitrary cut off, <0.2 AND >0.8 in both tissues (liver and kidney), also including genes which are unmethylated in both tissues (==0). Triangle dots in the bottom right are genes methylated in liver and not in kidney, whereas square dots in the upper left are genes methylated in kidney and not in liver and in the scatter plot origo is a cluster of genes unmethylated in both tissues.

The substantially differentiated gene methylation by value comparing the two organs indicates organ specialization and maturation in progress.

A series of candidate genes of potential importance to follow were displayed, some of which are transcription factors involved in gene expression and thus also related to development and organ/tissue differentiation and maturation (Homeobox family Zinc finger proteins), heat and cold shock proteins(HIF) and stress responders to light, hypoxia and low temperature (CIRBP), sex development (gonadotropin releasing hormone, gnrh1), various transmembrane molecules, interferons and other immune genes, insulin like receptor, phosphatases, myogenic regulatory factors.

From the reduced gene pool and based on data from both tissues, 46 genes were recovered belonging to the Hoxgene family which also were mostly hypomethylated and strongly deviating from the global methylation pattern, as well as also partly differentially distributed tissue wise. The data is depicted in FIG. 4. The figure is a scatter plot of a collection of 46 genes belonging to the Hox gene family, large black dots, based on data from both tissues and from the total pool (53 715) of genes. These genes are fundamental transcription factors in early life development and in organ/tissue, cell differentiation and hence many of which are relevant for targeting optimization of production regimes in aquaculture together with abovementioned candidate genes, not the least the seed or smolt production phase in salmonids.

TABLE 4
Genes methylated in Liver, and “not” in Kidney (Triangle dots in FIG. 3).

| Liver | Kindey | ID | Gene | |
|---|---|---|---|---|
| 1.0000000 | 0.14285714 | gene3706 | mrpl17 | ribosomal protein L17 mitochondrial-like protein(mrpl17) |
| 1.0000000 | 0.08823529 | gene4577 | il4k | 14 kDa transmembrane protein(i14k) |
| 1.0000000 | 0.16666667 | gene12265 | tm149 | Transmembrane protein 149(tm149) |
| 0.8260870 | 0.00000000 | gene13294 | rs2 | 40S ribosomal protein S2(rs2) |
| 0.8571429 | 0.00000000 | gene13712 | tmem220 | transmembrane protein 220(tmem220) |
| 0.9062500 | 0.03571429 | gene20850 | mpnd | MPN domain containing(mpnd) |
| 0.8730159 | 0.00000000 | gene23797 | gfm2 | G elongation factor mitochondrial 2(gfm2) |
| 0.8846154 | 0.00000000 | gene25993 | slc25a20 | solute carrier family 25 member 20(slc25a20) |
| 0.8333333 | 0.00000000 | gene27005 | ccdc66 | coiled-coil domain containing 66(ccdc66) |
| 1.0000000 | 0.11111111 | gene28172 | caco1 | Calcium-binding and coiled-coil domain-containing |
| | | | | protein 1(caco1) |
| 1.0000000 | 0.07894737 | gene29578 | esm1 | endothelial cell specific molecule 1(esm1) |
| 0.8636364 | 0.00000000 | gene33201 | pqlc2 | PQ loop repeat containing 2(pqlc2) |
| 0.8125000 | 0.16666667 | gene33932 | fance | Fanconi anemia complementation group E(fance) |
| 0.8285714 | 0.00000000 | gene46543 | rilpl2 | Rab interacting lysosomal protein like 2(rilpl2) |
| 0.8205128 | 0.00000000 | gene46778 | asb6 | ankyrin repeat and SOCS box containing 6(asb6) |
| 1.0000000 | 0.18750000 | gene47180 | mcidas | multiciliate differentiation and DNA synthesis associated |
| | | | | cell cycle protein(mcidas) |

TABLE 5
Genes methylated in kidney, “not” in liver (square dots in FIG. 3)

| Liver | Kindey | ID | Gene | |
|---|---|---|---|---|
| 0.11111111 | 0.9230769 | gene533 | tmed8 | transmembrane p24 trafficking protein family member 8 |
| 0.00000000 | 0.9166667 | gene693 | gnpi | Glucosamine-6-phosphate isomerase(gnpi) |
| 0.00000000 | 0.8750000 | gene2971 | znf79 | zinc finger protein 79(znf79) |
| 0.00000000 | 1.0000000 | gene3561 | rpp38 | ribonuclease P/MRP subunit p38 |
| 0.00000000 | 1.0000000 | gene4934 | junb | jun B proto-oncogene(junb) |
| 0.00000000 | 0.8461538 | gene6005 | grk7 | G protein-coupled receptor kinase 7(grk7) |
| 0.08333333 | 0.8157895 | gene6098 | tsen15 | tRNA splicing endonuclease subunit 15 |
| 0.15000000 | 0.8823529 | gene9597 | sat1 | spermidine/spermine N1-acetyltransferase 1 |
| 0.00000000 | 1.0000000 | gene11625 | s22a4 | Solute carrier family 22 member |
| 0.00000000 | 0.9545455 | gene14632 | heca | hdc homolog, cell cycle regulator(heca) |
| 0.13793103 | 0.9657534 | gene15744 | nelfa | negative elongation factor complex member A |
| 0.15384615 | 0.8750000 | gene18576 | gar1 | GAR1 ribonucleoprotein(gar1) |
| 0.00000000 | 0.8750000 | gene19026 | rnf103 | ring finger protein 103(rnf103) |
| 0.00000000 | 1.0000000 | gene20089 | lyrm9 | LYR motif containing 9(lyrm9) |
| 0.00000000 | 0.9230769 | gene21048 | mgdp1 | Magnesium-dependent phosphatase 1 |
| 0.17647059 | 0.8943171 | gene21244 | cpt2 | carnitine palmitoyltransferase 2(cpt2) |
| 0.00000000 | 1.0000000 | gene22357 | lamb1 | laminin subunit beta 1(lamb1) |
| 0.00000000 | 1.0000000 | gene22420 | cssa10h12orf29 | chromosome ssa10 open reading frame, |
| | | | | human C12orf29 |
| 0.04761905 | 0.9666667 | gene24538 | rab1a | RAB1A, member RAS oncogene family |
| 0.00000000 | 0.8142857 | gene25917 | pck1 | phosphoenolpyruvate carboxykinase 1 |
| 0.14705882 | 1.0000000 | gene28331 | srxn1 | sulfiredoxin 1(srxn1) |
| 0.04878049 | 0.9166667 | gene32369 | hlx | H2.0-like homeobox protein(hlx) |
| 0.12500000 | 0.9111333 | gene32437 | cssa15h6orf165 | |
| 0.00000000 | 1.0000000 | gene33231 | masp2 | mannan binding lectin serine peptidase 2 |
| 0.06666667 | 0.8477069 | gene33391 | mk13 | Mitogen-activated protein kinase 13 |
| 0.16666667 | 1.0000000 | gene33858 | ssrd | Translocon-associated protein subunit delta |
| 0.16666667 | 0.9171935 | gene34795 | ccl8 | C—C motif chemokine 8(ccl8) |
| 0.00000000 | 0.8909091 | gene36214 | lig4 | DNA ligase 4(lig4) |
| 0.00000000 | 1.0000000 | gene37997 | fuom | fucose mutarotase(fuom) |
| 0.00000000 | 1.0000000 | gene39610 | c1ql3 | complement C1q like 3(c1ql3) |
| 0.00000000 | 1.0000000 | gene39798 | enosf1 | enolase superfamily member 1(enosf1) |
| 0.13333333 | 0.8750000 | gene39883 | map10 | microtubule associated protein 10(map10) |
| 0.13793103 | 0.8421053 | gene40747 | fen1 | flap structure-specific endonuclease 1(fen1) |
| 0.00000000 | 0.8174762 | gene43528 | tbccd1 | TBCC domain containing 1 |
| 0.00000000 | 0.8055556 | gene44160 | yod1 | YOD1 deubiquitinase(yod1) |
| 0.00000000 | 1.0000000 | gene44446 | cdk2 | cyclin dependent kinase 2(cdk2) |
| 0.00000000 | 1.0000000 | gene44737 | cssa22h1orf74 | chromosome ssa22 open reading frame, |
| | | | | human C1orf74 |
| 0.00000000 | 1.0000000 | gene46434 | mrps27 | mitochondrial ribosomal protein S27 |
| 0.15000000 | 0.8648649 | gene46459 | znf131 | zinc finger protein 131(znf131) |
| 0.00000000 | 0.8454545 | gene47125 | ppm1f | protein phosphatase, Mg2+/Mn2+ dependent 1F |
| 0.00000000 | 0.8461538 | gene49799 | rnf139 | ring finger protein 139(rnf139) |
| 0.05555556 | 1.0000000 | gene50507 | bet1 | Bet1 golgi vesicular membrane trafficking protein(bet1) |
| 0.00000000 | 0.8750000 | gene72192 | heatr6 | HEAT repeat containing 6(heatr6) |
| 0.00000000 | 0.9000000 | gene65810 | atg7 | autophagy related 7(atg7) |
| 0.00000000 | 1.0000000 | gene76347 | tmem243 | transmembrane protein 243(tmem243) |

TABLE 6
Genes unmethylated in both kidney and liver (dots with methylation value == 0 in FIG. 3)

| Liver | Kindey | ID | Gene | |
|---|---|---|---|---|
| 0 | 0 | gene721 | foxg1 | forkhead boxG1(foxg1) |
| 0 | 0 | gene1306 | hmx3 | H6 family homeobox 3(hmx3) |
| 0 | 0 | gene2271 | rxfp3 | relaxin/insulin like family peptide receptor 3(rxfp3) |
| 0 | 0 | gene4031 | gbp | GSK-3-binding protein(gbp) |
| 0 | 0 | gene4304 | med18 | mediator complex subunit 18(med18) |
| 0 | 0 | gene7224 | ifne | interferon epsilon(ifne) |
| 0 | 0 | gene9385 | pcf11 | PCF11 cleavage and polyadenylation factor subunit(pcf11) |
| 0 | 0 | gene17941 | itpk1 | inositol 1,3,4-triphosphate 5/6 kinase(itpk1) |
| 0 | 0 | gene20324 | wbs18 | Williams-Beuren syndrome chromosomal region 18 protein |
| | | | | homolog(wbsl8) |
| 0 | 0 | gene20680 | fam43a | family with sequence similarity 43 member A(fam43a) |
| 0 | 0 | gene23918 | lage3 | L antigen family member 3(lage3) |
| 0 | 0 | gene24328 | yk001 | UNQ655/PRO1286(yk001) |
| 0 | 0 | gene25235 | ier2 | immediate early response 2(ier2) |
| 0 | 0 | gene26504 | necp2 | Adaptin ear-binding coat-associated protein 2(necp2) |
| 0 | 0 | gene30825 | tceanc2 | transcription elongation factor A N-terminal and central domain |
| | | | | containing 2(tceanc2) |
| 0 | 0 | gene31029 | cart | cocaine- and amphetamine-regulated transcript(cart) |
| 0 | 0 | gene31260 | rprd2 | regulation of nuclear pre-mRNA domain containing 2(rprd2) |
| 0 | 0 | gene33726 | hoxc12ab | homeobox protein HoxC12ab(hoxc12ab) |
| 0 | 0 | gene35287 | polr2h | RNA polymerase II subunit H(polr2h) |
| 0 | 0 | gene37439 | mrf4 | myogenic regulatory factor 4(mrf4) |
| 0 | 0 | gene38081 | mb21d1 | Mab-21 domain containing 1(mb21d1) |
| 0 | 0 | gene39521 | cbln2 | cerebellin 2 precursor(cbln2) |
| 0 | 0 | gene40990 | gnrh1 | gonadotropin releasing hormone 1(gnrh1) |
| 0 | 0 | gene44480 | hoxc12 | homeobox C12(hoxc12) |
| 0 | 0 | gene45052 | cl012 | CL012 protein(cl012) |
| 0 | 0 | gene46513 | cirbp | cold inducible RNA binding protein(cirbp) |
| 0 | 0 | gene47091 | fem1c | fem-1 homolog C(fem1c) |
| 0 | 0 | gene47483 | hoxd9aa | homeobox protein HoxD9aa(hoxd9aa) |
| 0 | 0 | gene48652 | tha11 | THAP domain-containing protein |
| 0 | 0 | gene48897 | arp19 | cAMP-regulated phosphoprotein 19(arp19) |
| 0 | 0 | gene73640 | bt2a2 | Butyrophilin subfamily 2 member A2(bt2a2) |
| 0 | 0 | gene81022 | rnd3 | Rho-related GTP-binding protein RhoE(rnd3) |
| 0 | 0 | gene62060 | ub2v1 | Ubiquitin-conjugating enzyme E2 variant 1(ub2v1) |
| 0 | 0 | gene71740 | zdh11 | Probable palmitoyltransferase ZDHHC11(zdh11) |
| 0 | 0 | gene78979 | rp19 | ribosomal protein L9(rpl9) |

### Example 2

### Comparison of Epigenetic Signatures with Performance

The applicant has performed studies wherein epigenetic signatures were obtained and analysed, e.g. as shown in Example 1, and comparing and correlating these with fish production regimes (protocols) and with performance data records (growth rate, health and carcass qualities). Reference is made to the description, Table A and B and FIGS. 6 to 10.

1. A method to identify fish characteristics of farmed fish, wherein the method comprises the steps of:
i) sampling to obtain fish sample material;
ii) DNA sequencing, comprising carrying out genome sequencing of the fish sample material;
iii) analysing the revealed genome data set of step ii) and establishing epigenetic signatures for the sample material; and optionally
iv) comparing and correlating the epigenetic signatures obtained with existing epigenetic signatures;
wherein the prepared epigenetic signatures of the fish sample material are for use as authenticators for fish.

2. The method of claim 1 wherein the fish sample material is taken from, or its signature reflects, either of; Genome; Gene; Organ, tissue or cell; or the life phase of the fish sample.

3. The method of claim 1 wherein the fish sample material is from any of individuals, organs, tissues or blood from any of the stages of a fish' life-cycle, comprising either of fertilized eggs, larvae, fry, parr and individuals at smoltification stages, from the sea grow-out phase until harvest or post-harvest, or organs, tissues, or blood from any of these.

4. The method of claim 1 wherein the prepared epigenetic signature forms part of an epigenetic signature data bank.

5. The method of claim 1 comprising the step iv) of comparing the epigenetic signature of one sample material with existing epigenetic signatures.

6. The method of claim 1 further comprising
a step of comparing the epigenetic signatures of one or more first group of fish with epigenetic signatures representing data characteristics for traits and performance of one or more other group of fish.

7. The method of claim 1 further comprising the step of correlating the epigenetic signatures of a fish sample material, and optionally gene expression profiles or transcriptome profiles of this, to performance data for fish.

8. The method of claim 7 wherein the performance data comprise data for either of life phase, fish management regimes and protocols, traits and performance, sea phase grow out performance, or post-harvest characteristics.

9. The method of claim 1 wherein the epigenetic signatures are used in traceability of fish, as a verification of a given fish production protocol/regime, or in determination of the origin of escaped farmed fish.

10. The method of claim 1, wherein the method is for distinguishing between different production regimes, distinguishing between smolt with different potentials for sea phase grow-out performance, or for predicting the resulting sea phase performance.

11. The method of claim 1 wherein the method is for use in providing feedback, such as to the hatchery operators, to assist in optimizing the fish production protocols and regimes, such as the smolt production regimes, and/or the sea phase production regimes.

12. The method of claim 1 wherein the method is for verification of the quality of the fish.

13. The method of claim 1 wherein the method is for determining or verifying the degree of smolt maturation.

14. The method of claim 1 for assessment of smolt status, optimizing smolt production, preparation of quality smolt, or producing high yields and healthy smolt.

15. The method of claim 1, further comprising a step of preparing a global methylation graph reflecting an individual fish's relative maturation stage and age, or a tissue or organ specific methylation graph reflecting differentiation or maturation.

16. The method of claim 1 wherein the epigenetic signature, and particularly the methylation distribution of this, of CpG islands is analysed.

17. The method of claim 1, wherein at least one epigenetic signature is prepared for one or more of the genes from the group of i2c2, hepc1, cherp, lyric, daam2, cenph, henmt1, ptprc and gemin6.

18-19. (canceled)

20. The method of claim 1, wherein at least one epigenetic signature is prepared for one or more of the genes from the group of LOC106578259, i2c2, hepcl, LOC106561688, LOC106603928, LOC106582077, LOC106604853, LOC106572469, LOC106588302, LOC106571646, LOC106601362, LOC106584016, LOC106611981, LOC106589905, LOC106564914, LOC106565121, LOC100380863, LOC106600693, and kiaa1211.

21. The method of claim 1, wherein feedback from the prepared epigenetic signatures linked with performance data is for use in establishing and optimizing fish farming production regimes and corresponding protocols.

22. The method of claim 1, wherein the prepared epigenetic signatures form part of an epigenetic signature-based test system for one or more of the following: qualities of bony fish; robustness, maturation, biological age, chronological age, optimization of feed and feeding regimes, handling disease, and breeding regimes.
